(19) United States
(12) Patent Application Publication
(10) Pub. No.: US 2007/0123881 A1
(43) Pub. Date: May 31, 2007

(54) Off-set bone plates

(76) Inventors: James D. Ralph, Bethlehem, PA (US); Stephen L. Tatar, Montville, NJ (US); Thomas N. Troxell, Pottstown, PA (US)

Correspondence Address: Norris, McLaughlin & Marcus, P.A., 721 Route 202-206, P.O. Box 1018, Somerville, NJ, 08879-1018, US

(21) Appl. No.: 11/259,709

(22) Filed: Oct. 26, 2005

(51) Int. Cl. A61F 2/30

(52) U.S. Cl. 606/69

(57) ABSTRACT

Longitudinal plates and center plates for a bone plate assembly which comprise one or more protrusions. The bone plate assemblies comprise longitudinal plates and optional center plates with connecting rods and further have observation windows and protrusions. Also, methods for treating broken bones of a patient by applying the longitudinal plates and center plates.

### BACKGROUND OF THE INVENTION

1. Field of the Invention

The invention pertains to bone plates, and longitudinal plates and center plates that are components of the bone plates, which have off-set sections. The longitudinal plates and center plates which, in part, form the structure of the bone plates have at least one end which contains a protrusion, and when the longitudinal plates and center plates are joined together by connecting rods, the protrusions of adjacent longitudinal plates and center plates are mutually opposed, and the protrusions, in part, stop excess twisting of the bone plate when applied to the broken bone of a patient. The bone plates further comprise one or more observation windows which allow a surgeon to observe the location of a broken bone while moving the pieces of broken bone together.

2. The Related Art

Conventional bone plates have fixed dimensions and multiple holes for accommodating bone screws. Surgeons generally use bone plates to join sections of fractured bone by placing the bone plate atop the fracture, inserting bone screws through the holes in the plate which overlie the healthy part of the bone and securing the bone about the fracture. Bone plates of many sizes are provided for the surgeon, each having a number of holes so that the surgeon can arrange the plate over the fracture and have bone screw holes available above the healthy bone.

DCP dates are also available to stabilize fractured bone. The DCP plates generally have angulated openings, through which screws are “toed” into the bone with the tightening of the toed screws operating to move the bone, slightly, under the DCP plate. DCP plates have been used to move bone pieces on either side of the fracture closer together. However, with a DCP plate, only slight and not easily controllable movements occur. Generally, the underlying bone is pulled into place by the threads of a screw, providing no control over the twisting or turning of the bone, and the bending of the plate. In addition, the bone is pulled at an angle to the attached plates, which can result in a cocked bone, or at an angle to the longitudinal direction of the bone. This angulation, together with the micromotion in the bone, has lead to backing out of the screws after the bone plate is secured to the bone.

Thus, there is a need for bone plates, such as bone plate assemblies which are adjustable, that allow the surgeon to move the broken pieces of bone while inhibiting or preventing the bone from twisting or cocking either during the process of applying the bone plate to the fractured bone or afterwards. Providing the bone plate with one or more windows to allow the surgeon to observe the pieces of fractured bone moving together while the bone plate is adjusted on the bone would also be desired.

The invention pertains to a bone plate which comprises longitudinal plates and, optionally, center plates which have at least one end comprising a protrusion. The longitudinal plates and center plates form a bone plate assembly by the use of connecting rods which allows the surgeon to move the longitudinal plates and center plates and, thus, the bone to which they are attached generally longitudinally prior to fixing the longitudinal length for the bone plate while providing one or more windows for the surgeon to observe the pieces of fractured bone coming together. In the assembled bone plate, the protrusion of one longitudinal plate or center plate is juxtaposed with the protrusion of an adjacent longitudinal plate or center plate thereby establishing, within the window area of the bone plate, an off-set section which provides structural strength to the bone plate and inhibits or prevents twisting and turning of the longitudinal plates and center plates, and the bone plate itself, when the bone plate is being applied to a patient and after application.

### SUMMARY OF THE INVENTION

The bone plates, i.e. the bone plate assemblies, of the invention comprise longitudinal plates and, optionally, center plates which comprise two ends with at least one end having a protrusion at either the distal side and/or forward side of the longitudinal plate or center plate. The protrusion has an inward wall and an outward wall which are about parallel to each other and the protrusion further comprises an end wall which extends, from the ends of the inward wall and opposite outward wall. The protrusion further comprises an upper surface and a lower surface, which are generally intergral with or part of the upper surface and lower surface of the longitudinal plate and/or the top surface and bottom surface of the center plate. The longitudinal plates further comprise a forward side, a distal side, a top surface and a bottom surface, and may be made a hollow piece or a solid piece. Likewise, the center plate generally comprises a forward longitudinal side, a distal longitudinal side, a first end, a second end, a top surface and a bottom surface, and may be a hollow piece or a solid piece. The longitudinal plates and center plates may further comprise at least one locking means bore defined by a locking means bore wall and at least one bone screw bore defined by a bone screw bore wall. In certain embodiments of the invention the locking means bore is not necessary.

The longitudinal plates and center plates also comprise at least one, such as two or more, rod bores. The rod bores may extend from rod bore openings at an end of the longitudinal plate and center plate and/or from the end wall of the protrusion to a point between the two ends of the longitudinal plate or center plate. The rod bores are defined by rod bore inner walls and rod bore ends. In an embodiment of the invention, however, the rod bores may be continuous, extending from one end to the opposite end, particularly in center plates, in which case the rod bores are defined by at least rod bore walls. The rod bore walls may be any geometrical shape but preferably are continuous walls forming a cylindrical shape to accommodate connecting rods. In an embodiment of the invention, the rod bores comprise threaded rod bore walls or the rod bore walls comprise threaded sections.

The bone plate assembly comprises one or more longitudinal plates and, optionally, one or more center plates and one or more connecting rods. The connecting rods are generally cylindrical elements having a first end and a second end with a continuous outer surface there between. The connecting rods translate within the rod bores to form the bone plate assembly such that the longitudinal plates and center plates are capable of movement by translating with the connecting rods thereby providing the bone plates with adjustability, generally in a longitudinal direction. The connecting rods have an axial dimension that is not completely within the rod bores and, thus, the connecting rods and ends of the longitudinal plates and center plates define one or more windows within the bone plate assembly which allows the surgeon to see the bone coming together under the bone plate when applying the bone plate to a patient. In certain embodiments of the invention, the connecting rods comprise one or more threaded ends, or have slots or comprise one or more retaining pin holes.

The protrusions of adjacent longitudinal plates and/or center plates within the bone plate assembly may be mutually opposed or have surfaces, such as inward walls, juxtaposed with one another thereby establishing an off-set section adjacent to, or preferably within, the window area. This off-set arrangement of the protrusions provides structural strength to the bone plate and inhibits or prevents twisting and turning of the longitudinal plates and center plates, and the bone plate itself, when the bone plate is being applied to a patient and after application.

### DETAILED DESCRIPTION OF THE DRAWINGS

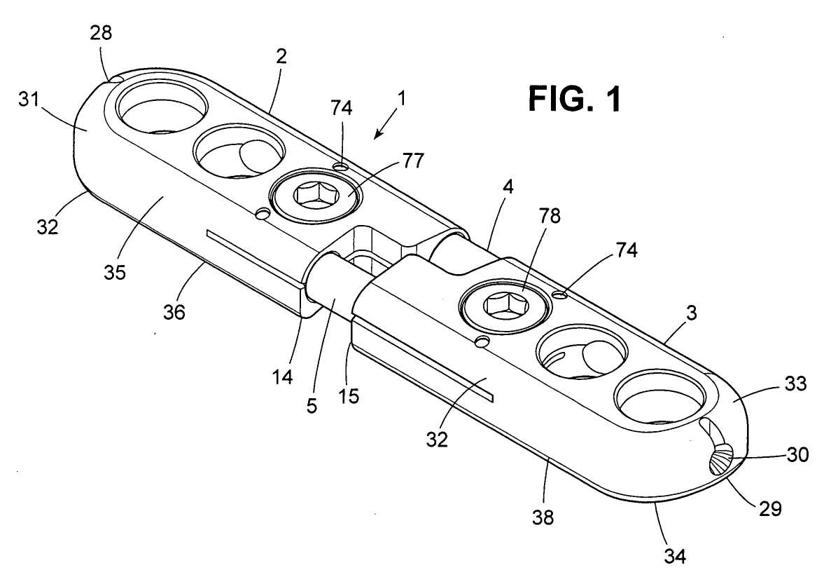

FIG. 1 is a top perspective view of a bone plate assembly according to an embodiment of the invention.

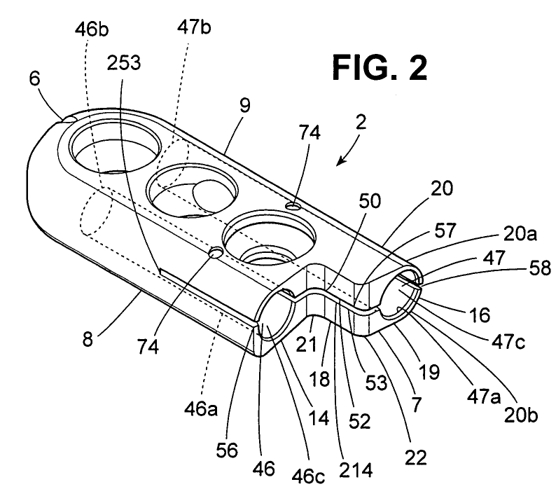

FIG. 2 is a top perspective view of a longitudinal plate according to an embodiment of the invention.

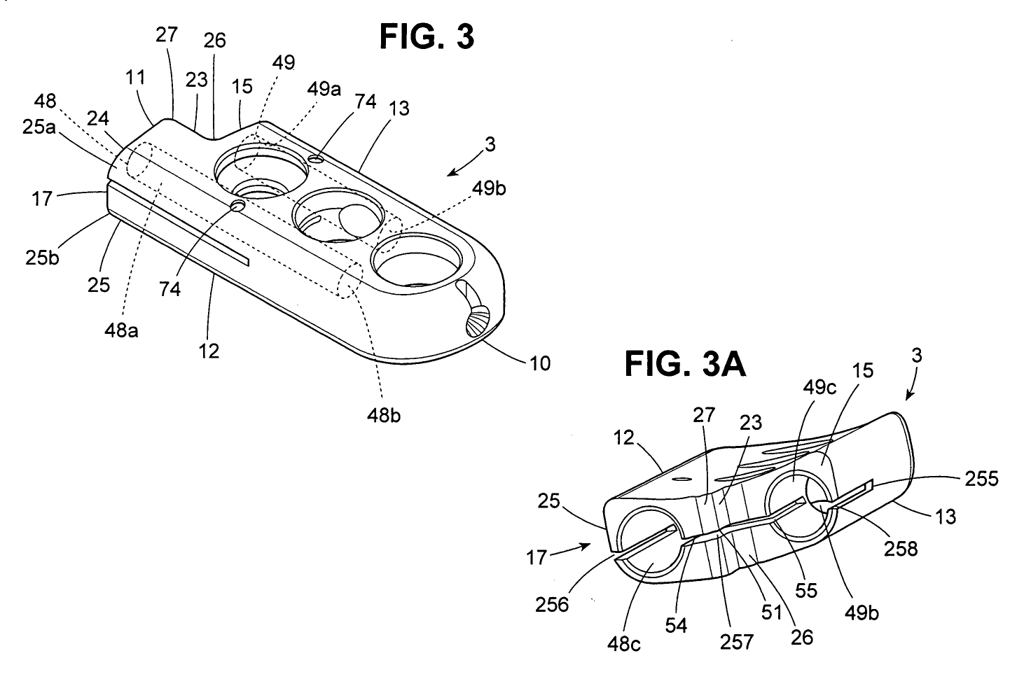

FIG. 3 is a top perspective view of a longitudinal plate according to an embodiment of the invention.

FIG. 3A is a perspective end view of the longitudinal plate shown in FIG. 3.

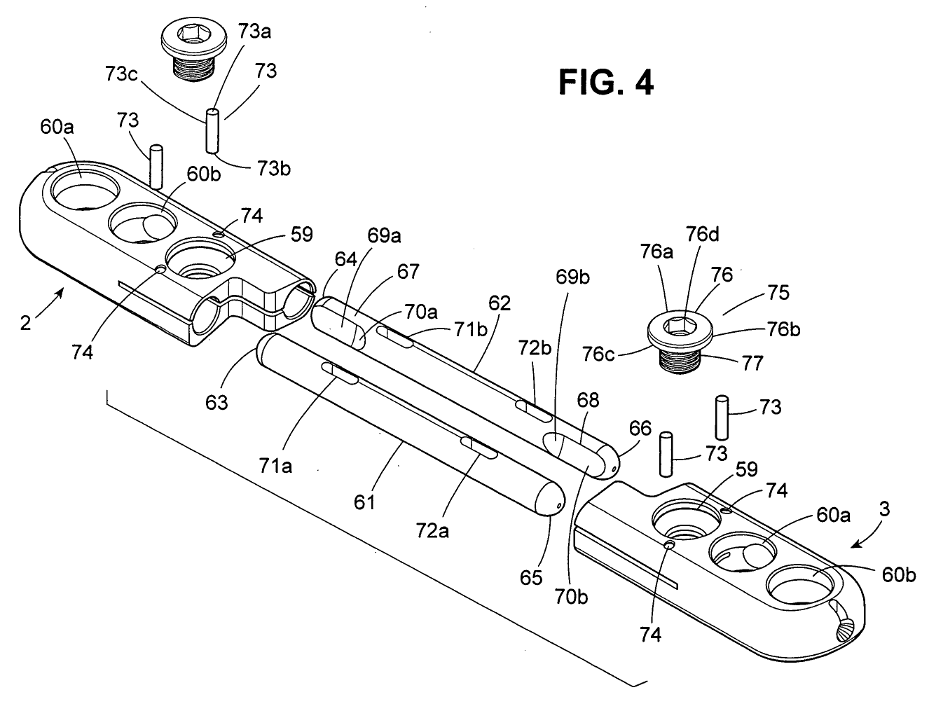

FIG. 4 is a perspective exploded view of the bone plate assembly of FIG. 1.

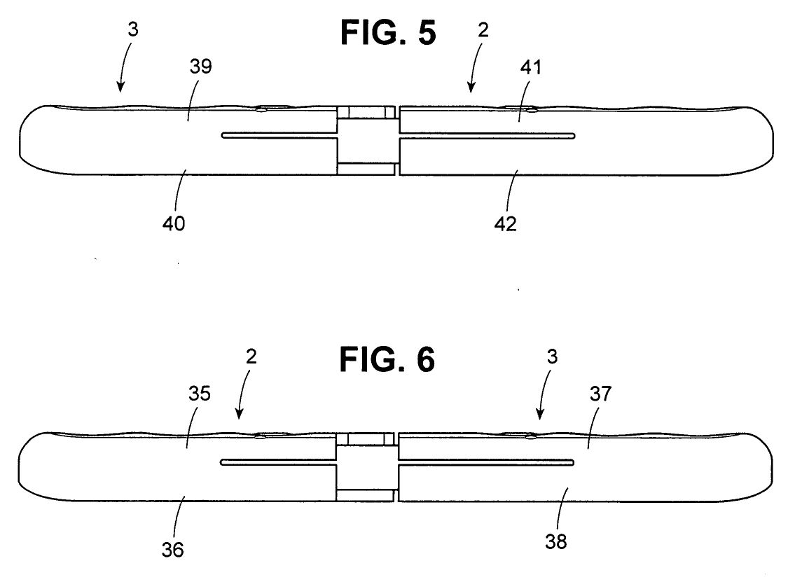

FIG. 5 is a side view of a bone plate assembly according to an embodiment of the invention.

FIG. 6 is a side view (opposite to the side view of FIG. 5) of a bone plate assembly according to an embodiment of the invention.

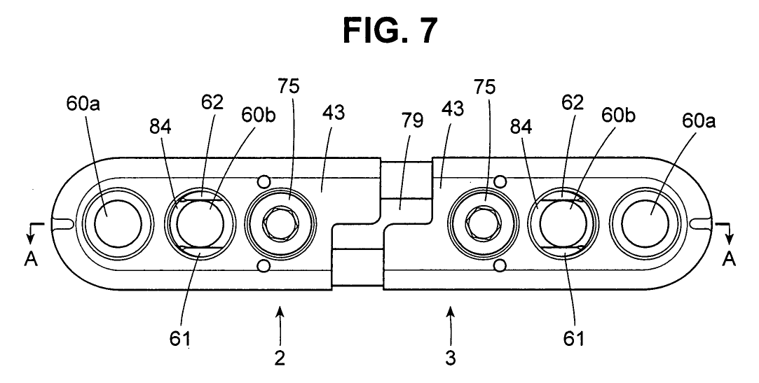

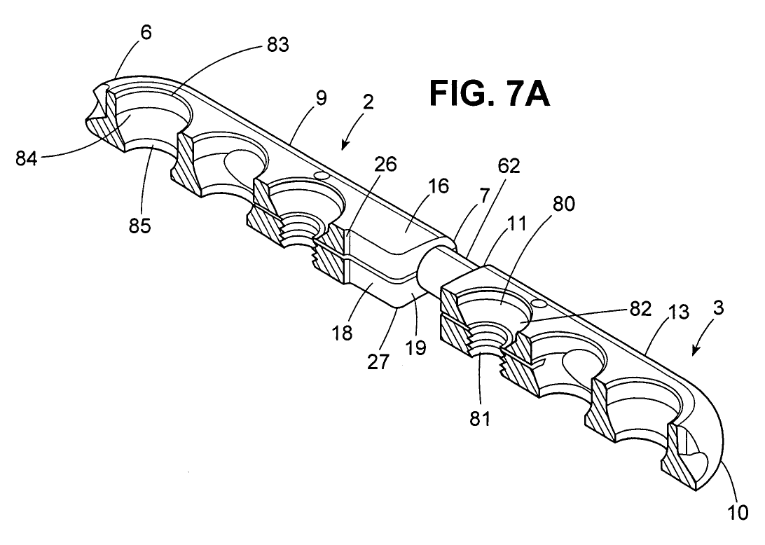

FIG. 7 is a top view of a bone plate assembly according to an embodiment of the invention.

FIG. 7A is a cross-section view of the bone plate assembly along line A-A′ of FIG. 7.

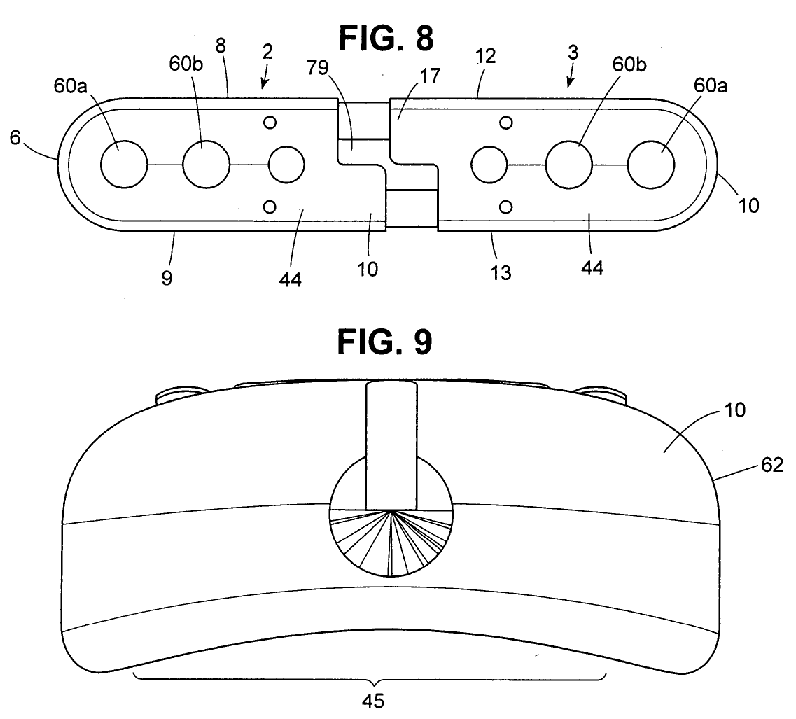

FIG. 8 is a bottom view of a bone plate assembly according to an embodiment of the invention.

FIG. 9 is a side view of a bone plate assembly according to an embodiment of the invention.

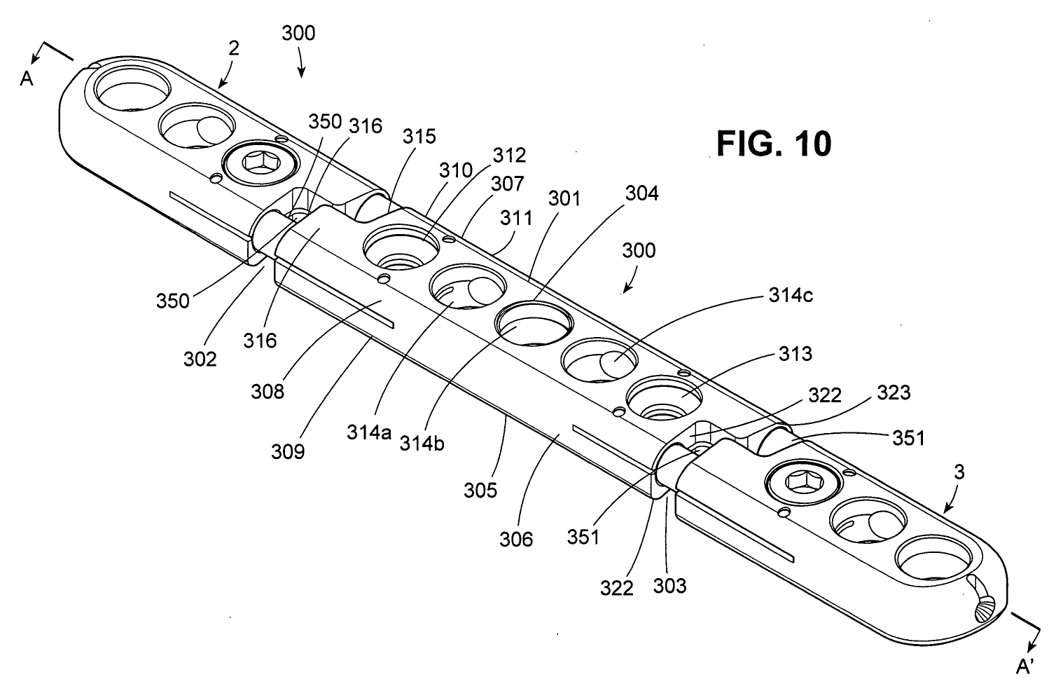

FIG. 10 is a top perspective view of a bone plate assembly according to an embodiment of the invention.

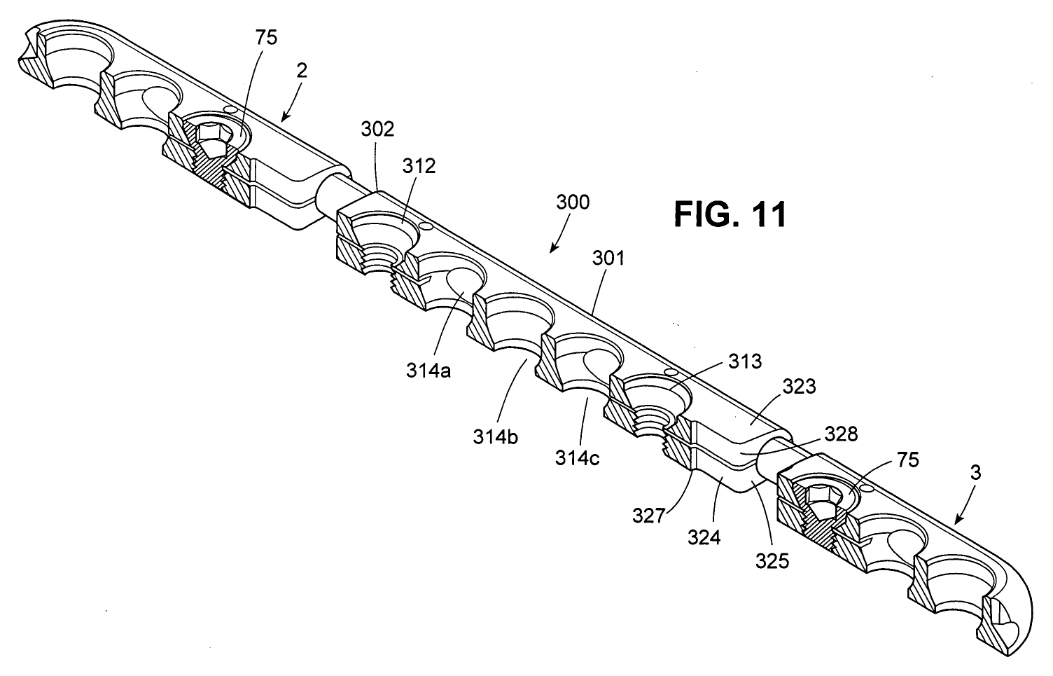

FIG. 11 is a cross-sectional view of the bone plate assembly shown in FIG. 10 along line A-A′.

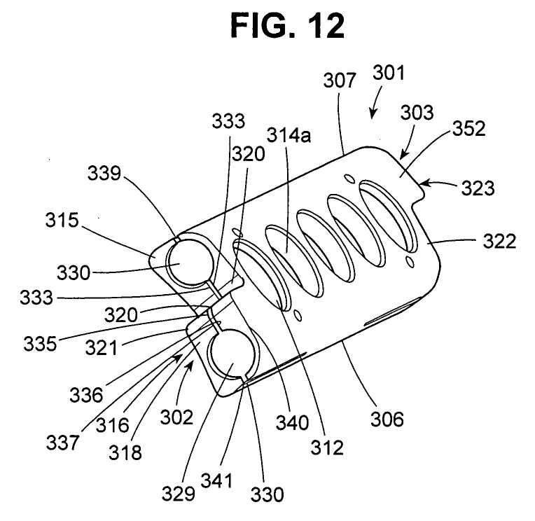

FIG. 12 is an end view of a bone plate assembly according to an embodiment of the invention.

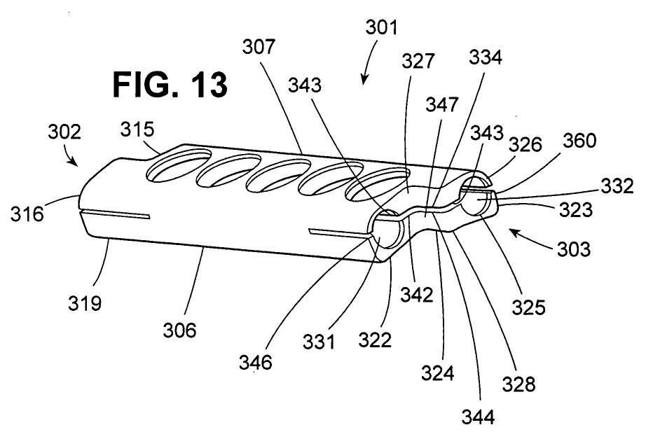

FIG. 13 is an end view of a bone plate assembly according to an embodiment of the invention.

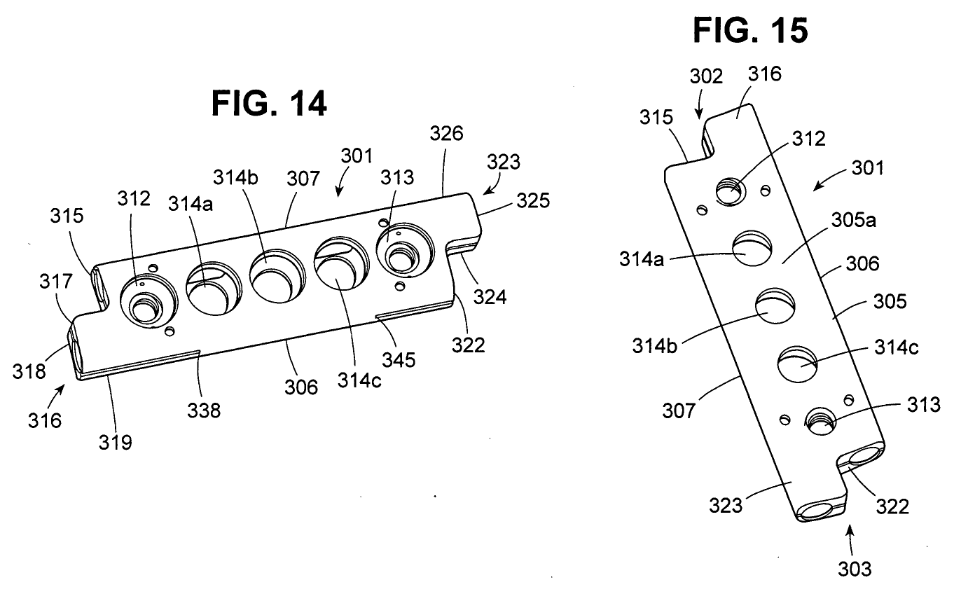

FIG. 14 is a top view of a bone plate assembly according to an embodiment of the invention.

FIG. 15 is a bottom view of a bone plate assembly according to an embodiment of the invention.

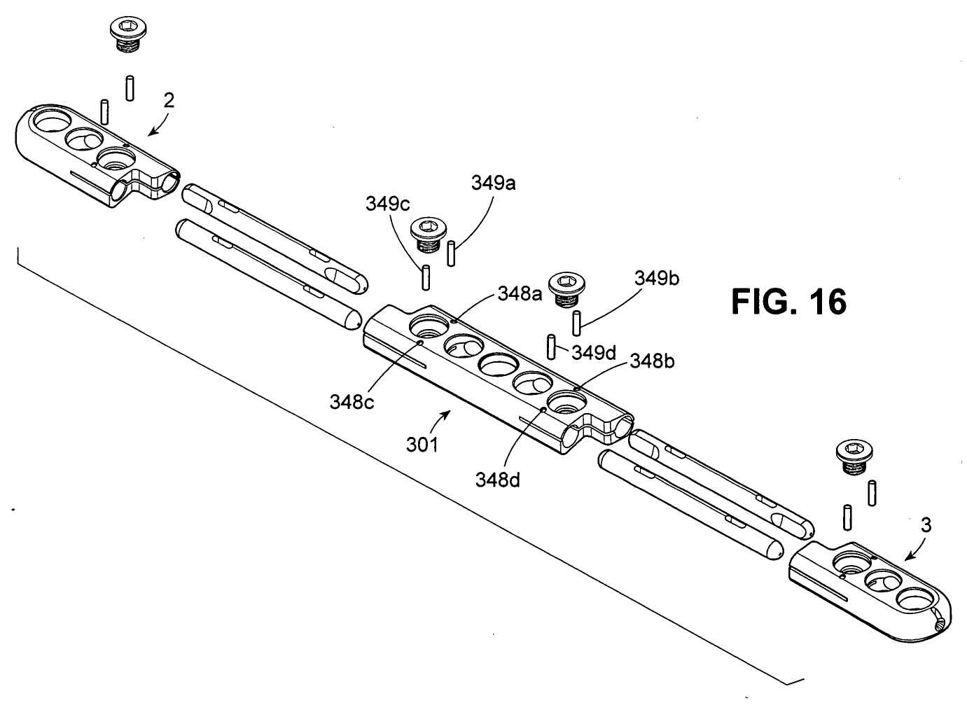

FIG. 16 is a perspective exploded view of the bone plate assembly of FIG. 10.

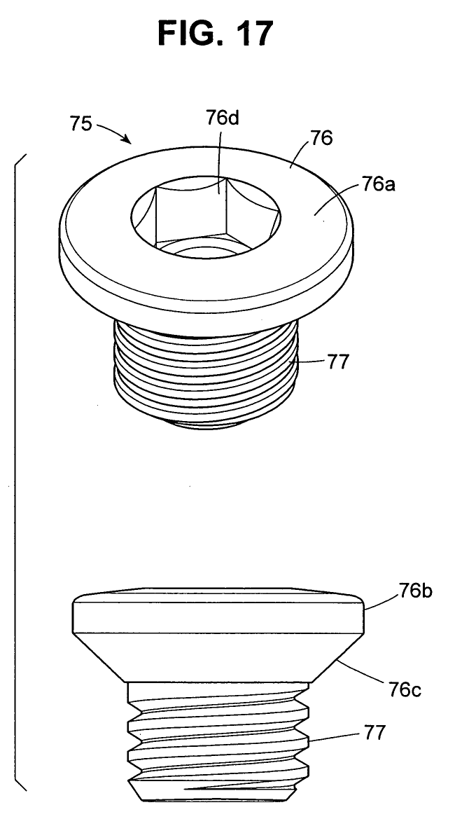

FIG. 17 is a perspective view of locking means in accordance with an embodiment of the invention.

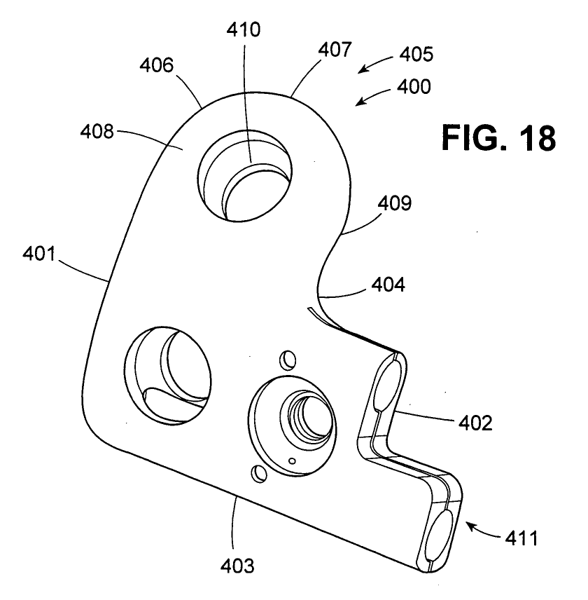

FIG. 18 is a perspective view of a longitudinal plate in accordance with an embodiment of the invention.

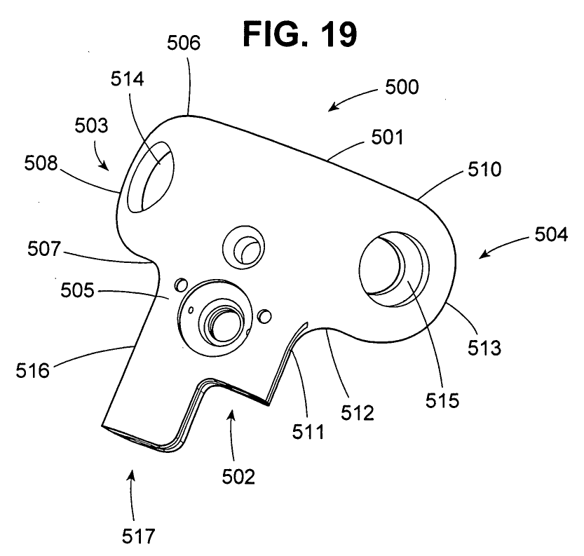

FIG. 19 is a perspective view of a longitudinal plate in accordance with an embodiment of the invention.

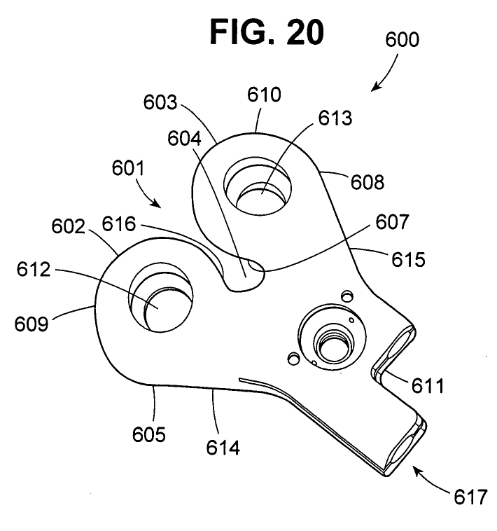

FIG. 20 is a perspective view of a longitudinal plate in accordance with an embodiment of the invention.

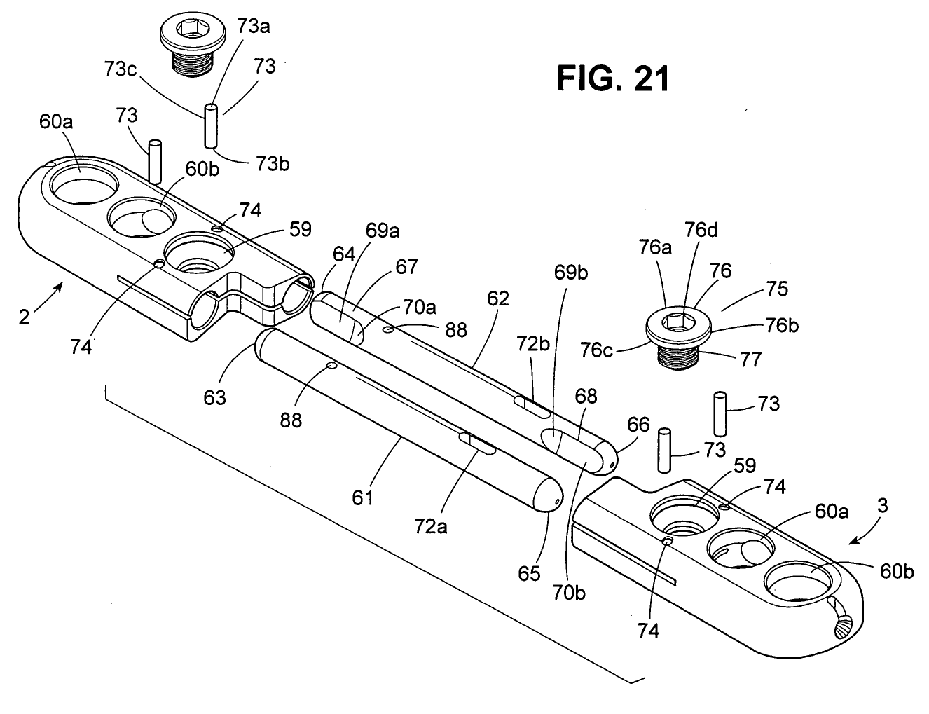

FIG. 21 is an exploded perspective view of a bone plate assembly in accordance with an embodiment of the invention wherein the connecting rods comprise retaining pin holes.

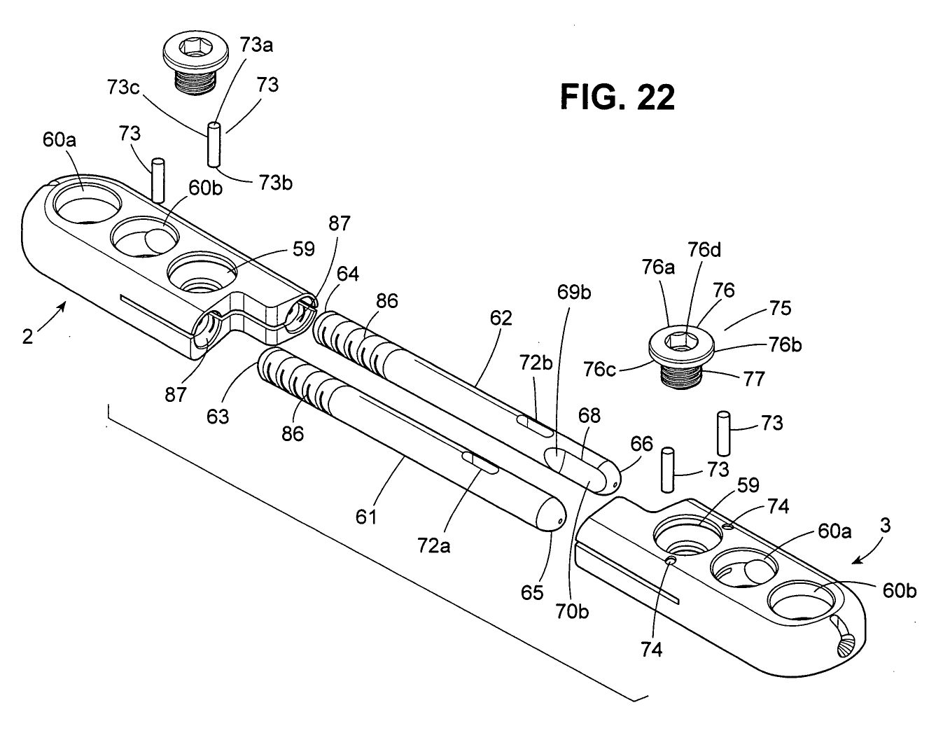

FIG. 22 is an exploded perspective view of a bone plate assembly in accordance with an embodiment of the invention wherein the connecting rods have threaded ends.

### DETAILED DESCRIPTION OF THE INVENTION

FIG. 1 illustrates a bone plate 1 in accordance with an embodiment of the invention comprising a first longitudinal plate 2, a second longitudinal plate 3, a distal connecting rod 4 and a forward connecting rod 5. As shown FIG. 2, the first longitudinal plate 2 has a first end 6, a second end 7, a forward side 8 and a distal side 9. The second longitudinal plate, as shown in FIG. 3, also comprises a first end 10, a second end 11, a forward side 12 and a distal side 13.

The second end of each of the first longitudinal plate and second longitudinal plate comprises a recessed edge (shown as 14 with respect to the first longitudinal plate and 15 with respect to the second longitudinal plate) and a protrusion (shown as 16 with respect to the first longitudinal plate and 17 with respect to the second longitudinal plate). Each protrusion comprises an inward wall adjacent to the recessed edge, an end wall adjacent to the inward wall, which may be about perpendicular, or perpendicular to the inward wall, and an outward wall which is adjacent to the end wall, with the outward wall generally parallel to the inward wall. The outward walls of the protrusions are generally extensions of the forward side or distal side, depending on the side of the longitudinal plate that the protrusion is located. As shown in FIG. 2, for example, the protrusion 16 of the first longitudinal plate comprises an inward wall 18, an end wall 19 and an outward wall 20. There may be a lower curved section 21, having generally concave curvature between the recessed edge and the inward wall and an upper curved section 22 having generally convex curvature between the inward wall and end wall. Likewise, as shown in FIG. 3, for example, the protrusion 17 of the second longitudinal plate comprises an inward wall 23, an end wall 24 and an outward wall 25. There may be a lower curved section 26, having generally concave curvature between the recessed edge and the inward wall and an upper curved section 27 having generally convex curvature between the inward wall and end wall. The curved sections of the protrusions provide a continuous curved shape thereby avoiding sharp edges at the contact points of the walls and edges.

As shown in the figures, such as FIGS. 1-3, the first end (6, 10) of each longitudinal plate may have lateral curvature having an upper arc point (shown as 28 for the first longitudinal plate and 29 for the second longitudinal plate) about equidistant from the forward side and distal side of the longitudinal plates. The first end of each longitudinal plate optionally comprises a locking slot (shown in FIG. 1 for example in the second longitudinal plate as 30) at about the upper arc point. Also, the first end of both the first longitudinal plate and the second longitudinal plate comprise a first end upper longitudinally curved surface (shown as 31 for the first longitudinal plate and 33 for the second longitudinal plate) and a first end lower longitudinally curved surface (shown as 32 for the first longitudinal plate and 34 for the second longitudinal plate).

Again referring to FIGS. 1-3, the forward side of each longitudinal plate comprises a forward side upper longitudinally curved surface (shown as 35 for the first longitudinal plate and 37 for the second longitudinal plate) and a forward side lower longitudinally curved surface (shown as 36 for the first longitudinal plate and 38 for the second longitudinal plate). Also, the distal side of each longitudinal plate comprises a distal side upper longitudinally curved surface (shown as 39 for the first longitudinal plate and 41 for the second longitudinal plate) and a distal side lower longitudinally curved surface (shown as 40 for the first longitudinal plate and 42 for the second longitudinal plate). Also, the outward wall of the protrusion on each longitudinal plate comprises an outward wall upper longitudinally curved surface (shown in FIG. 2 as 20a and FIG. 3 as 25a) and an outward wall lower longitudinally curved surface (shown in FIG. 2 as 20b and FIG. 3 as 25b). For each longitudinal plate, the forward side upper longitudinally curved surface, forward side lower longitudinally curved surface, the distal side upper longitudinally curved surface, distal side lower longitudinally curved surface, outward wall upper longitudinally curved surface and outward wall lower longitudinally curved surface together with the first end upper longitudinally curved surface and a first end lower longitudinally curved surface provide each longitudinal plate, and the bone plate assembly, with an uninterrupted longitudinal curve. Also, the longitudinal and lateral surfaces allow the bone plates to fit snugly against the curvature of the bone structure.

Each longitudinal plate, particularly as shown in FIGS. 7 and 8, have an upper surface 43 and a lower surface 44. The upper surface and lower surface of the longitudinal plates extend over the protrusions and form upper and lower surfaces for the protrusion. The lower surface generally comprises a concave arcuate section 45 (particularly as shown in FIG. 9) which further facilitates the snug fit of the bone plate against the curvature of the bone structure.

Each longitudinal plate comprises two or more rod bores which are defined by rod bore walls and rod bore ends. For example, as shown in FIG. 2, the first longitudinal plate 2 has a forward rod bore 46 defined by a forward rod bore wall 46a and a forward rod bore end 46b with a forward rod bore opening 46c at the recessed edge of the second end and a distal rod bore 47 defined by a distal rod bore wall 47a and a distal rod bore end 47b and a distal rod bore opening 47c at the end wall of the protrusion. The forward rod bore 46 of the first longitudinal plate extends longitudinally from the recessed edge to a point between the recessed edge of the second end and the first end of the first longitudinal plate and the distal rod bore 47 of the first longitudinal plate extends longitudinally from the end wall of the protrusion at the second end to a point between the end wall and the first end of the second longitudinal plate. As show in FIGS. 3 and 3a the second longitudinal plate 3 has a forward rod bore 48 at the end wall of the protrusion defined by a forward rod bore wall 48a and a forward rod bore end 48b with a forward rod bore opening 48c at the end wall of the protrusion and a distal rod bore 49 at the recessed edge of the second end defined by a distal rod bore wall 49a and a distal rod bore end 49b with a distal rod bore opening 49c at the recessed edge. The forward rod bore 48 of the second longitudinal plate extends longitudinally from the end wall of the protrusion at the second end to a point between the end wall and the first end of the second longitudinal plate and the distal rod bore 49 of the second longitudinal plate extends longitudinally from the recessed edge to a point between the recessed edge of the second end and the first end of the first longitudinal plate. Some or all of the rod bores may be threaded in that the rod bore walls comprise a threaded section, generally proximate to the second end, and the entire rod bore wall may be threaded.

Each longitudinal plate further comprises a slit, except in the embodiments of the invention wherein the connecting rods are permanently affixed to all rod bores in the longitudinal plate, such as by use of threaded connecting rods and rod bore walls that comprise a threaded section and by use of connecting rods with retaining pin holes used in conjunction with retaining pins, as described in more detail below. As shown in FIG. 2 with respect to the first longitudinal plate, the second end comprises a slit opening 50 which extends from the forward side to the distal side along the recessed edge, the inward wall of the protrusion and the end wall of the protrusion. As shown in FIG. 3a, the second longitudinal plate 3 also comprises a slit opening 51 which extends from the forward side to the distal side along the recessed edge, the inward wall of the protrusion and the end wall of the protrusion. The slits of each longitudinal plate extend longitudinally from its respective slit opening at the second end to a point between the second end and the first end of each longitudinal plate. The slit of each longitudinal plate has a slit upper wall (shown in FIG. 2 for the first longitudinal plate as 52 and in FIG. 3a for the second longitudinal plate as 54) and a slit lower wall (shown in FIG. 2 for the first longitudinal plate as 53 and in FIG. 3a for the second longitudinal plate as 55) and the gap between the slit upper wall and a slit lower wall to the slit end (shown in FIG. 2 as 253 for the first longitudinal plate and in FIG. 3a as 255 for the second longitudinal plate) for each longitudinal plate defines the slit of each respective longitudinal plate.

The slit of each of the first longitudinal plate and/or second longitudinal plate optionally comprises three sections. For example, as shown in FIG. 2 with respect to the first longitudinal plate, the slit 214 may comprise a forward section 56 which has a forward opening at the forward side 8 and extends to a point on the forward bore wall thereby providing a gap in the forward bore wall with the gap between the slit upper wall 52 and a slit lower wall 53 from the forward side to the forward rod bore to the slit end 253 defining the forward section 56 of the slit. The second section 57 is the central section which extends from an opening on the forward rod bore, opposite to the point where the forward section intersects the forward rod bore, to a point on the distal rod bore. Thus, the gap between the slit upper wall 52 and a slit lower wall 53 from the forward rod bore to the distal rod bore to the slit end 253 defines the central section 57 of the slit. The third section, the distal section 58 of the slit, extends from an opening on the distal rod bore, opposite to the point where the central section intersects the distal rod bore, to a point on the outward wall of the protrusion and the distal wall. Thus, the gap between the slit upper wall 52 and a slit lower wall 53 from the distal rod bore to the distal side of the longitudinal plate to the slit end 253 defines the distal section 58 of the slit 214. The second longitudinal plate may also optionally comprise a three section slit similar to that discussed above. As shown in FIG. 3a with respect to the second longitudinal plate, the slit 215 may comprise a forward section 256 which has a forward opening at the outward side 25 of the protrusion and forward side 8 and extends to a point on the forward bore wall thereby providing a gap in the forward bore wall with the gap between the slit upper wall 54 and a slit lower wall 55 from the forward side to the forward rod bore to the slit end 255 defining the forward section 256 of the slit. The second section is the central section 257 which extends from an opening on the forward rod bore, opposite to the point where the forward section intersects the forward rod bore, to a point on the distal rod bore. Thus, the gap between the slit upper wall 54 and a slit lower wall 55 from the forward rod bore to the distal rod bore to the slit end 255 defines the central section 257 of the slit. The third section, the distal section 258 of the slit, extends from an opening on the distal rod bore, opposite to the point where the central section intersects the distal rod bore, to a point on the distal wall. Thus, the gap between the slit upper wall 54 and a slit lower wall 55 from the distal rod bore to the distal side of the longitudinal plate to the slit end 255 defines the distal section 258 of the slit of the second longitudinal plate 215. The slit is preferably along the midline of each longitudinal plate, the midline being the point about halfway between the top surface and a longitudinal plane of the lowest point on the lower surface of each longitudinal plate.

Each longitudinal plate may further comprise one or more locking means bores defined by locking means bore walls and one or more bone screw bores defined by bone screw bore walls. In a preferred embodiment of the invention, the locking means bores are spherical and have a threaded section and the bone screw bores are spherical for angulation of the bone screws and have smooth surfaces, e.g. no threading. In the embodiment of the invention shown in the drawings, each longitudinal plate comprises one locking means bore 59 and two bone screw bores 60a and 60b. As shown in the drawings, the slit of each longitudinal plate extends from the second end through the locking means bores and terminates proximate to one of the bone screw bores preferably the bone screw bore proximate to the locking means bore.

The longitudinal plates are assembled into a bone plate by use of connecting rods which translate within the rod bores. The connecting rods in the bone plates may have different longitudinal lengths. In the embodiment of the invention shown in FIGS. 1-9, the bone plate comprises two connecting rods, a forward connecting rod 61 and a distal connecting rod 62. The connecting rods are generally cylindrical in shape having a curved continuous outer surface, a first end (shown in FIG. 4 as 63 for the forward connecting rod and 64 for the distal connecting rod) and an opposite second end (shown in FIG. 4 as 65 for the forward connecting rod and 66 for the distal connecting rod). Each connecting rod, at both the first end and second end, has a recessed section. The recessed sections are shown in FIG. 4 as a first end recessed section 67 and a second end recessed section 68 with respect to the distal connecting rod, however, any of the connecting rods in the bone plates of the invention may have such recessed sections. Each recessed section generally has a concave section 69a and 69b and an adjacent cut in section 70a and 70b. In an embodiment of the invention, as shown in FIG. 22, at least one end of one or more connecting rods in the bone plate assembly comprise threaded ends 86 which mate with threaded rod bores 87 of longitudinal plates, i.e. threading on part or all of the rod bore inner walls, to affix the connecting rods to the longitudinal plates, and/or center plates.

The connecting rods may optionally comprise one or more slots. The slots generally extend from a point on the surface of each rod to an opposite point on the surface and are an opening within the rod defined by a slot inner surface. As shown in the drawings, particularly FIG. 4, each rod optionally has a first slot 71a and 71b and a second slot 72a and 72b. The slots may be aligned at a defined angle to the plane of the recessed sections.

The bone plate may optionally comprise one or more retaining pins. As shown in FIG. 4, for example, the retaining pins 73 are cylindrical elements having a first end 73a, a second end 73b and a continuous outer surface 73c between the ends. Each longitudinal plate may optionally, as shown in the drawings, particularly FIGS. 1-4, comprise one or more retaining pin bores 74 which are defined by retaining pin bore walls which extend vertically from the upper surface of each longitudinal plate to the lower surface of each longitudinal plate or with center plates from the top surface to the bottom surface, and generally bisect the connecting rod bores. The retaining pin bores may be proximate to the locking means bores or are otherwise located proximate to the second end of the longitudinal plate in line with the connecting rod bores.

The retaining pins may have threaded sections. For example, the continuous outer surface of the retaining pin may be smooth or may have threads either at the first end or second end of the retaining pin, at both the first end and second end of the retaining pin or along the entire continuous outer surface of the retaining pin. Threading of the retaining pin facilitates intermixing of the longitudinal plates and/or center plates by the surgeon when treating a patient with broken bone(s). Likewise, the retaining pin bores may be threaded either at each end, both ends or along the entire retaining pin bore wall to interact with threaded retaining pins.

In an embodiment of the invention, as shown in FIG. 21, the connecting rods comprise one or more retaining pin holes 81 proximate to one or more ends of the connecting rods which are defined by retaining pin hole walls that extend from one point on the outer surface of the connecting rod to another point on the connecting rod, and the retaining pin hole may be angled. The retaining pin holes can be aligned with the retaining pin bores of the longitudinal plates or center plates in which event the retaining pin may be translated through the retaining pin bore and retaining pin hole to affix the connecting rod to a longitudinal plate or center plate. The retaining pin hole and/or the retaining pin bore may be threaded to mate with a threaded retaining pin.

FIGS. 4 and 17 show a locking means which may be used in accordance with an embodiment of the invention. The locking means comprises a large-top set screw, 75 having a top section 76 and a threaded section 77. The top section 76 has a breadth, i.e. diameter, greater than the diameter of the threaded section 77. The top section has an upper surface 76a, an upper section 76b having a diameter and a frustoconical section 76c which has a diameter that tapers from the diameter of the upper surface and upper section to the diameter of the threaded section. The locking means may further have a recessed section 76d to accommodate a tool for tightening the locking means.

Referring now to FIG. 7A, the locking means bores in the longitudinal plates, optionally, each have an upper segment 80 and a threaded segment 81. The upper segment has a larger dimension than the threaded segment and the locking means bores further comprise the frustoconical segment 82 having a dimension that tapers from the dimension of the upper segment to the dimension of the threaded segment. The locking means bores are defined by locking means bore walls, including walls of the upper segment 80, threaded segment 81 and a frustoconical segment 82. As discussed above, the locking means bores are preferably spherical. The dimensions of the upper segment 80, frustoconical segment 82 and lower segment 81 complement the dimensions of the large top set screw such that the upper segment corresponds to the upper section of the large top set screw, the frustoconical segment corresponds to the frustoconical section of the large top set screw and the threaded segment corresponds to the threaded section of the large top set screw so that the large top set screws when secured into the locking means bores, are about flush with the top surface of the longitudinal plate, i.e. the large top set screw is recessed into the longitudinal plate. Likewise, as shown in FIG. 7A, each bone screw bore is defined by bone screw bore walls, including walls of the segments in that each bone screw bore comprises an upper segment 83, frustoconical segment 84 and lower segment 85, with the dimension of the frustoconical segment tapering from that of the upper segment to that of the lower segment, which complements the shape of the bone screw similar to that discussed above for the large top set screw and locking means bore. Thus, when a bone screw is inserted into the bone screw bore, the top surface of the bone screw is flush, or about flush, with the upper surface of the longitudinal plate such that the bone screws are recessed into the longitudinal plate. Also, the bone screws will lock with the bone screw bores and also the bone of the patient. As discussed above, the bone screw bores preferably have smooth surfaces and are spherical shaped to facilitate angulation of the bone screws.

In the embodiment of the invention shown in FIGS. 1-9, the assembled bone plate comprises a first longitudinal plate 2, a second longitudinal plate 3, two connecting rods (a distal rod 4 and forward rod 5) and two large top set screws, as described above (a first large top set screw designated as element 77 in FIG. 1 and a second large top set screw designated as element 78 in FIG. 1). The bone plate assembly may also, optionally, comprise four retaining pins. The bone plate may be assembled by inserting the first ends of the connecting rods into the bore holes of the first longitudinal plate and the second ends of the connecting rods into the bore holes of the second longitudinal plate of the bone plate. When assembled, as shown in the drawings, for example FIG. 7, the bone plate assembly has the first longitudinal plate and second longitudinal plate with opposed second ends, and the gap between the opposed second ends framed by the connecting rods defines the observation window 79.

When the connecting rods have retaining pin holes or threaded ends, the connecting rods may be effectively secured, i.e. affixed, within the forward rod bore and/or distal rod bore of the first longitudinal plate or second longitudinal plate and then the opposite ends of the connecting rods may be slid into the rod bores of the corresponding plate to which the connecting rods are not effectively secured. Alternatively, one end of one of the connecting rods may be effectively secured within the distal rod bore of the first longitudinal plate and one end of the other connecting rod may be effectively secured within the forward rod bore of the second longitudinal plate, or vice versa, and then the free ends of the connecting rods can be slid in the vacant rod bores of the opposite longitudinal plate, and similar sequencing can be used when the bone plate assembly comprises center plates which are discussed below. For example, a connecting rod may be placed in the distal rod bore of the first longitudinal plate leaving the forward rod bore vacant and a second connecting rod may be placed in the forward rod bore of the second longitudinal plate leaving the distal rod bore vacant, and when the longitudinal plates are brought together, the connecting rod in the distal rod bore of the first longitudinal plate is slid into the distal rod bore of the second longitudinal plate and the connecting rod in the forward rod bore of the second longitudinal plate is slid into the forward rod bore of the first longitudinal plate, and, likewise, a connecting rod can be placed in the forward rod bore of the first longitudinal plate and a second connecting rod can be placed in the distal rod bore of second longitudinal plate to similarly assemble the bone plate. In these, and other, embodiments, one or more ends of the connecting rod may be threaded to mate with a threaded rod bore and, also, the connecting rod may have one or more retaining pin holes to mate with retaining pins which translate through retaining pin bores in the longitudinal plates and/or center plates, and the retaining pins and retaining pin holes and retaining pin bores may be threaded.

In the embodiment shown in FIGS. 1-9, the protrusion of the first longitudinal plate is on the distal side of the longitudinal plate and the protrusion of the second longitudinal plate is on the forward side of the second longitudinal plate such that when the first longitudinal plate and second longitudinal plate are moved closer to one another the inward wall of the protrusion of the first longitudinal plate and inward wall of the protrusion of the second longitudinal plate become juxtaposed within the window which allows the bone plate to have enhanced rigidity within the window by providing a nearly continuous bridging section within the window formed by the area where the protrusions of the longitudinal plates off-set, i.e. have juxtaposed inward walls, while maintaining a window within the center of the plate to allow the surgeon to observe the bone being moved together when applying the bone plate to a patient. Thus, the off-set protrusions inhibit the bone plate and bone from twisting during application and use during the patient healing process and beyond.

The longitudinal plates may have alternate configurations proximate to or as part of the first end of the longitudinal plate, such as an “L-shape”, “T-shape” and ‘Y-shape”, as described with respect to the first ends of certain longitudinal plates described in the inventors' co-pending patent application entitled ADJUSTABLE BONE PLATE filed on Oct. 24, 2005 under the Patent Cooperation Treaty in the United States Receiving Office (serial number not yet assigned) which is a Continuation-in-Part of U.S. patent application Ser. No. 10/975,296 filed Oct. 28, 2004. Both of these applications are incorporated by reference herein in their entirety.

As shown in FIG. 18, the longitudinal plate 400 having the “L-shaped” configuration comprises a first end 401, a second end 402, a forward side 403, a distal side 405 and a bump out 406, which is proximate to the first end. The bump out 406 comprises curvature 407 which extends from the distal termination point 408 of the first end to the distal termination point 409 of the distal side 405. The longitudinal plate 400 may comprise a bone screw hole 410 proximate to the bump out 407. Longitudinal plate 400 further comprises the protrusion 411 at the second end 402.

FIG. 19 shows another embodiment wherein the longitudinal plate 500 has a “t-shaped” configuration. In this embodiment, the longitudinal plate comprises a first end 501 and a second end 502 with mutually opposed lobes proximate to the first end, the forward lobe 503 and distal lobe 504. The forward lobe 503 is adjacent to the first end and forward side 516 and extends from the forward termination point 505 of the first end to the termination point 506 of the forward side. The forward lobe comprises a forward lobe first curved section 507 which is concave and adjacent thereto a forward lobe second curved section 508 which is convex. The distal lobe 504 is adjacent to the first end and distal side 509 and extends from the distal termination point 510 of the first end to the termination point 511 of the distal side. The distal lobe 504 comprises a distal lobe first curved section 512 which is concave and adjacent thereto a distal lobe second curved section 513 which is convex. A forward lobe bone screw bore 514 which extends from the upper surface to the lower surface is proximate to the forward lobe, and a distal lobe bone screw bore 515 which extends from the upper surface to the lower surface is proximate the distal lobe. The curvature of the longitudinal plate 500 allows the surgeon to place the longitudinal plate over the curvature of a broken bone. Because the longitudinal plate has curvature, when bone screws are inserted into the forward lobe bone screw bore 514 and distal lobe bone screw bore 515, the ends of the bone screws within the bone point towards each other, e.g., toe, which inhibits twisting of the bone plate and provides greater assurance that the bone screws will not pull out of the bone. Longitudinal plate 500 further comprises the protrusion 517 at the second end 502.

FIG. 20 shows an embodiment wherein the longitudinal plate has a “Y-shaped” configuration. In this embodiment, the longitudinal plate 600 comprises a first end 601 which is comprised of a forward circular element 602 and a distal circular element 603 joined by a center section 604 having curvature. The forward circular element has a continuous side 609 which extends from a first forward termination point 605 to a second forward termination point 616 such that the continuous side 609 is circular or semi-circular in orientation. Likewise, the distal circular element has a continuous side 610 which extends from a first distal termination point 607 to a second distal termination point 608, such that the continuous side 610 is circular or semi-circular in orientation. The center section is adjacent to the second forward termination point 604 and the first distal termination point 607, and extends from the second forward termination point 616 to the first distal termination point 607. The longitudinal plate 600 comprises a forward side edge 614 extending from the first forward termination point 605 to the second end 611, and a distal side edge 615 extending from the second distal termination point 608 to the second end 611. A forward bone screw bore 612 is proximate to the forward circular element 602 and a distal bone screw bore 613 is proximate to the distal circular element 603. The curvature of the longitudinal plate 600 allows the surgeon to place the longitudinal plate over the curvature of a broken bone. Because the longitudinal plate has curvature, when bone screws are inserted into the forward bone screw bore 612 and distal bone screw bore 613, the ends of the bone screws within the bone point towards each other, e.g., toe, which inhibits twisting of the bone plate and provides greater assurance that the bone screws will not pull out of the bone. Longitudinal plate 600 further comprises the protrusion 617 at the second end 611.

In an embodiment of the invention, as shown in FIGS. 10, 11 and 16, the bone plate assembly 300 comprises a center plate 301 for receiving one or more connecting rods. The center plate may receive connecting rods from adjoining center plates, as shown, or in other embodiments of the invention, the bone plate assembly may comprise connecting rods which extend through the center plate and interact with adjoining center plate(s) and/or longitudinal plate(s). The bone plate 300 comprises the first longitudinal plate and second longitudinal plate and other elements associated with the bone plate, i.e., the locking means, optional retaining pins and such, as depicted, which are described above with respect to the embodiment of the invention shown in FIGS. 1-9.

Referring to FIGS. 10-16, the center plate has a first end 302 and a second end 303. The center plate 300 has a top surface 304 and a bottom surface 305. The center plate further comprises a forward longitudinal side 306 and distal longitudinal side 307. Similar to the sides of the longitudinal plates, the forward longitudinal side of the center plate comprises a forward center plate upper longitudinally curved surface 308 and a forward center plate lower longitudinally curved surface 309, and the distal longitudinal side of the center plate comprises a distal center plate upper longitudinally curved surface 310 and a distal center plate lower longitudinally curved surface 311. As discussed above, the curved shape of the longitudinal and lateral surfaces allow the bone plates to fit snugly against the curvature of the bone structure. The bottom surface of the center plate also generally comprises a concave arcuate section 305a at the bottom surface 305 which further facilitates the snug fit of the bone plate against the curvature of the bone structure.

Referring to FIGS. 10-16, the center plate generally comprises at least two, such as two, locking means bores, a first locking means bore 312 proximate to the first end 302 and a second locking means bore 313 proximate to the second end 303. The first and second locking means bore of the center plate are the same shape and have similar features as the locking means bores described above with respect to the longitudinal plates. The center plate further comprises one or more, such as three bone screw bores 314a (first end bone screw bore), 314b (mid bone screw bore) and 314c (second end bone screw bore) which are laterally between the first locking means bore and second locking means bore and have the same shape and configuration as described above with respect to the bone screw bores of the longitudinal plates. In embodiments of the invention wherein the connecting rods comprise threaded ends to mate with thread rod bores in the center plates or retaining pin holes used in conjunction with retaining pin bores in the center plate and retaining pins to affix one more connecting rods, the center plate may not comprise a locking means bore, proximate to one or both ends of the center plate and, further, may not have a slit at one or both ends of the center plate, particularly in embodiments wherein all connecting rods at one or both ends of the center plate may be affixed to one or both ends of the center plate or through continuous rod bores, either by threaded connecting rods/rod bores and/or the use of connecting rods having retaining pin holes in conjunction with retaining pin bores in the center plate and retaining pins.

The center plate comprises at least one protrusion at either the first end or second end. As such, at least either the first end or the second end may have a recessed edge and a protrusion. In an embodiment of the invention, each end of the center plate comprises a recessed edge and a protrusion. Each protrusion comprises an inward wall adjacent to the recessed edge, an end wall adjacent to the inward wall, which may be about perpendicular, or perpendicular to the inward wall, and an outward wall which is adjacent to the end wall, with the outward wall generally parallel to the inward wall and are generally extensions of the forward longitudinal side or distal longitudinal side.

Referring to the FIGS. 10-16, the first end 302 has a first end recessed edge 315 and a first end protrusion 316, with the protrusion at the forward side of the first end. The first end protrusion comprises a first end protrusion inward wall 317, a first end protrusion end wall 318 and a first end protrusion outward wall 319, with the outward wall being an extension of the forward longitudinal side 206 of the center plate. There may be a first end protrusion lower curved section 320, having generally concave curvature between the first end recessed edge and the first end protrusion inward wall and an first end protrusion upper curved section 321 having generally convex curvature between the first end protrusion inward wall and first end protrusion end wall. Referring again to the drawings, the second end 303 has a second end recessed edge 322 and a second end protrusion 323, with the protrusion at the distal side of the second end. The second end protrusion comprises a second end protrusion inward wall 324, a second end protrusion end wall 325 and a second end protrusion outward wall 326, with the second end protrusion outward wall being an extension of the distal longitudinal side 307 of the center plate. There may be a second end protrusion lower curved section 327, having generally concave curvature between the second end recessed edge and the second end protrusion inward wall and a second end protrusion upper curved section 328 having generally convex curvature between the second end protrusion inward wall and second end protrusion end wall. The curved sections of the protrusions provide a continuous curved shape thereby avoiding sharp edges at the contact points of the walls and edges. Further, each protrusion has a top surface and a bottom surface which are generally extensions of top surface 304 and bottom surface 305 of the center plate. Although the protrusions are shown in the drawings, FIG. 10, for example, at the forward side of the first end and distal side of the second end, the protrusions may be at either side provided that, when matched with either a longitudinal plate or other center plate, the protrusions among components of the assembled bone plate are mutually opposed, such as shown in the drawings.

The center plate comprises one or more rod bores, which are generally defined by at least rod bore walls and also may be further defined by rod bore ends. In an embodiment of the invention, the center plate may comprise continuous rod bores such as a forward continuous rod bore proximate to the forward side of the center plate having a first opening at the first end of the center plate and a second opening at the second end of the center plate. The forward continuous rod bore is generally defined by a forward continuous rod bore wall, which may be cylindrical in shape, which extends from the first opening to the second opening of the forward continuous rod bore. Likewise, in an embodiment of the invention, the center plate also comprises a distal continuous rod bore proximate to distal side of the center plate having a first opening at the first end of the center plate and a second opening at the second end of the center plate. The distal continuous rod bore is generally defined by a distal continuous rod bore wall, which may be cylindrical in shape, which extends from the first opening to the second opening of the distal continuous rod bore. One or more of the rod bores may be threaded, i.e. have all or part of the rod bore inner wall with threaded sections, to mate with threaded connecting rods, for example one rod bore at the first end of the center plate may be threaded and one rod bore at the second end of the center plate may be threaded, or all the rod bores of the center plate may be threaded, including embodiments comprising threaded continuous rod bores.

In the preferred embodiment of the invention, the center plate comprises two or more first end rod bores which extend from the first end to a point between the first end and second end, which are defined by first end rod bore walls and a first end rod bore end walls and having openings at the first end and two or more second end rod bores which extend from the second end to a point a point between the first end and second end which are defined by second end rod bore walls and second end rod bore end walls and having openings at the second end.

As shown in the drawings, particularly FIGS. 12 and 13, the first end forward rod bore 329 extends longitudinally from the first end of the center plate at the first end protrusion end wall 318 to proximate to the first end bone screw bore 314a next to the first locking means bore 312, and the first end distal rod bore 330 extends longitudinally from the first end of the center plate at the first end recessed edge to proximate to the first end bone screw bore 314a next to the first locking means bore 312. The second end forward rod bore 331 extends longitudinally from the second end of the center plate at the second end recessed edge 322 to proximate to the second end bone screw bore 314c next to the second locking means bore 313, and the second end distal rod bore 332 extends longitudinally from the second end of the center plate at the second end protrusion end wall 323 to proximate to the second end bone screw bore 314c next to the second locking means bore 313.

Except in the embodiments of the invention wherein the connecting rods are affixed to all rod bores at one or both ends of the center plate, the center plate comprises a first slit at the first end of the center plate and/or a second slit at the second end of the center plate. The first slit and second slit have the same shape, configuration and defining elements as described above with respect to the slit in the longitudinal plates.

As shown in FIGS. 10-16, the first slit 333 has a first slit opening 335 at the first end and is defined by first slit upper wall 336 and a first slit lower wall 337 and a first slit end 338. The gap between the first slit upper wall 336 and first slit lower wall 337 and the first slit end 338 defines the first slit 333. The first slit end 338 is generally proximate to the first end bone screw bore 314a closest to the first locking means bore 312. The first slit 333 is preferably along the midline of each center section, the midline being the point about halfway between the top surface and a longitudinal plane of the bottom surface of the center plate. Optionally, the first slit comprises three sections, the first a forward section 339 which has a forward opening at the first end protrusion outward wall 319 and the forward longitudinal side 306 and extends to a point on the first end forward rod bore 329, the second a central section 340 which extends from an opening on the first end forward rod bore, opposite to the point where the forward section intersects the first end forward rod bore to a point on the first end distal rod bore 330, and third, a distal section 341 which extends from an opening on the first end distal rod bore 330, opposite to the point where the central section intersects the first end distal rod bore, to a point on the distal longitudinal wall 307.

The second slit 334 has a second slit opening 342 at the second end and is defined by second slit upper wall 343 and a second slit lower wall 344 and a second slit end 345. The gap between the second slit upper wall 343 and second slit lower wall 344 and the second slit end 345 defines the second slit 334. The second slit end 345 is generally proximate to the second end bone screw bore 314c closest to the second locking means bore 313. The second slit 334 is preferably along the midline of each center section, the midline being the point about halfway between the top surface and a longitudinal plane of the lower surface 305 of the center plate. Optionally, the second slit comprises three sections, the first a forward section 346 which has a forward opening at the forward longitudinal side 306 and extends to a point on the second end forward bore 331, the second a central section 347 which extends from an opening on the second end forward bore, opposite to the point where the forward section intersects the second end forward bore to a point on the second end distal rod bore 332, and third, a distal section 360 which extends from an opening on the second end distal rod bore, opposite to the point where the central section intersects the second end distal rod bore, to the second end protrusion outward wall 326 and the distal wall 303.

In embodiments of the invention wherein the center-plate comprises a forward continuous rod bore and distal continuous rod bore, the sections of the first slit and second slit would be defined as discussed below, but with respect to the forward continuous rod bore and distal continuous rod bore as opposed to the first end forward rod bore, first end distal rod bore, second end forward rod bore and second end distal rod bore.

As shown in FIG. 16, the center plate may optionally comprise one or more, preferably four, retaining pin bores 348a-d, defined by retaining pin bore walls, and one or more, preferably four, retaining pins 349a-d. These retaining pin bores 348a-d and retaining pins 349a-d function the same as discussed above with respect to the longitudinal plates. The retaining pins and retaining pin bores may be threaded as discussed above with respect to the retaining pins and retaining pin bores for the longitudinal plates.

As shown in FIGS. 10, 11 and 16, the center plate may be used in conjunction with longitudinal plates to make up a bone plate. In the embodiment of the invention shown in the drawings, ends of connecting rods extending from the longitudinal plates are inserted into and translate within the connecting rod bore holes of the center plate to form a bone plate assembly 300, shown in FIG. 10 for example, having dual observation windows 350 and 351. This embodiment comprises the use of a second distal connecting rod and second forward connecting rod which connect between the center plate at the second end to the second longitudinal plate. Further, bone plates wherein connecting rods translate within continuous rod bores that extend beyond the first end and second end of the center plate and can translate within rod bores of longitudinal plates or other center plates to assemble a bone plate are within the scope of the invention.

When a bone is fractured in two places, the surgeon may place one longitudinal plate over one side of one of the breaks, the center plate over the bone between breaks and the other longitudinal plate over the bone on the other side of the second break. The plates would be secured to the bone with one or more bone screws in each of the longitudinal plates and in the center plate. The breaks can be brought together by moving the longitudinal plates and/or center plates in a direction to move the bone together, and then the longitudinal plates and/or center plates can be fixed in location by applying the large top set screw. The surgeon then applies more bone screws through available bone screw bores, if desired. Various lengths of connecting rods may be used to further accommodate breaks in two or more places in the bone of a patient, and also to accommodate use of the center plates comprising continuous connecting rod bores. It should be understood that one or more center plates may be used in which case the bone plate would have three or more observation windows. Thus, the invention encompasses plates having more than one center plate with two longitudinal plates which comprise two or more observation windows, or a number of observation windows equal to one plus the number of center plates. Bone plates having more than one center plate are particularly useful when used to treat multiple fractures by providing an observation window over each fracture area.

The ends of the center plates comprise protrusions and, when assembled as part of a bone plate with longitudinal plates and, optionally, other center plates, the protrusions at the ends of the center plates have inward walls that are juxtaposed with the inward walls of the protrusions of adjoining longitudinal plates and/or center plates. This arrangement provides the same benefits as discussed above with respect to the embodiment of the invention wherein the bone plate assembly comprises two longitudinal plates with off-set protrusions.

In an embodiment of the invention, the bone screws may further set the longitudinal plates, and when used, the center plate(s), in place by urging the connecting rods towards the distal side and/or forward side of the longitudinal plate or center plate. The recessed section of the connecting rods may be placed adjacent to, or, preferably, partially within one or more of the bone screws bores, preferably the upper segment of the bone screw bore and/or frustoconical segment of the bone screw bore such that the recessed section partially overlays the bone screw bore and when the bone screw is inserted into the bone screw bore and translates through the bone screw bore, the connecting rod proximate to the lateral side is urged towards lateral side and the connecting rod proximate to the distal side is urged towards the distal side thereby increasing frictional forces between the connecting rods and the inner surfaces of the connecting rod bores. For example, in FIG. 7, bone screw bores 60a on each of the first longitudinal plate and second longitudinal plate show the recessed section of the connecting rods (61, 62) overlaying the upper segment 83 and/or frustoconical segment 84 of these bone screw bores. As such, when the bone screw is inserted into the bone screw bore the distal connecting rod is urged towards the distal side of the longitudinal plate the forward connecting rod is urged towards the forward side of the longitudinal plate, thus increasing the frictional forces between the connecting rods and the inner surfaces of the connecting rod bores. FIG. 10 shows the first end bone screw bore 314a and second end bone screw bore 314c of the center plate as having the recessed section of connecting rods partially overlaying the bone screw bores, as discussed above.

Also, with respect to the large top set screws and bone screws, because the structure of the locking means bores and bone screws bores compliment the configuration of the large top set screws and bone screws, as discussed above, the large top set screw and bone screws are flush with the upper surface of the longitudinal plates and, when used, the center plate. Thus, the large top set screw and bone screw counter sink with the longitudinal plates and center plate and are recessed into the longitudinal plates and/or center plate(s). This will aid in precluding toggle and bone twisting.

In embodiments of the invention wherein both the distal connecting rod and forward connecting rod are both affixed to the same longitudinal plate or one or both ends of the center plate by either threaded connecting rods and/or connecting rods which comprise retaining pin holes for alignment with retaining pin bores in longitudinal plates or center plates which interact with retaining pins, all of which may be threaded, there is no need to use the locking means to releaseably secure the connecting rods to the longitudinal plate or center plate. Thus, in this embodiment of the invention, the locking means bore and slit, as well as the locking means, are not necessary for those particular longitudinal plates and center plates, or one or more ends of center plates, wherein two connecting rods are affixed. However, in embodiments wherein one connecting rod is affixed, either through connecting rods with threaded ends or with the use of the retaining pin hole and the other rod bore of the longitudinal plate or end of the center plate is vacant, then the longitudinal plate should comprise slit and locking means bore. With respect to the center plate, if both ends of the center plate have affixed connecting rods and no vacant rod bores then neither end would have a slit or locking means bore proximate thereto, if one end of the center plate has connecting rods affixed in both rod bores and the other end has at least one rod bore vacant then the end with vacant rod bore would comprise a slit and a locking means bore proximate thereto, and if both ends of the center plate have at least one vacant rod bore then both ends comprise the slit and a locking means bore proximate thereto.

The bone plates are generally assembled by translating the connecting rods within the connecting rod bores of the longitudinal plates and center plates. The locking means is inserted into the locking means bores of the longitudinal plates and, optionally, center plates and rotated such that the threaded section of the locking means mates with the threaded segment of the locking means bore causing the locking means to recess into the locking means bore as the locking means is moved in a direction. As the large top set screw moves downward, the upper wall of the slit is caused to move towards the lower wall of the slit thus creating a frictional force between the bore hole walls and the rods which restricts and precludes longitudinal movement of the longitudinal plates and center plates and/or the connecting rods thereby setting a longitudinal length of the bone plate. The lower wall of the slit may also be caused to move towards the upper wall fully or partially creating the frictional force. In effect, the locking mechanism crushes the slit and the locking means bores together to hold the longitudinal plates in fixed relationship to the rods. Because there is a gap between the ends of the longitudinal plates, and, if applicable, between the longitudinal plates and the center plates, these one or more gaps provide the surgeon with one or more windows to view the bone as he or she moves the bone plate together prior to selecting the appropriate therapeutic length.

The invention comprises a method for setting the broken bone of a patient. The method comprises providing one or more longitudinal plates and, optionally one or more center plates, providing one or more connecting rods, providing one or more locking means and providing one or more bone screws. The method further comprises securing at least a first longitudinal plate to the first side of the break in a bone of a patient by translating a bone screw through a bone screw bore and connecting the bone screw to the bone, inserting connecting rods into the connecting rod bores of the first longitudinal plate, placing a second longitudinal plate over the bone on the second side of a break in the bone of a patient and translating connecting rods through the bore holes in the second longitudinal plate, securing the second longitudinal plate to the bone by translating bone screws through a bone screw bore and connecting the bone screw to the bones, moving the longitudinal plates toward one another and providing and applying the locking means to set the length of the bone plate assembly. The method further comprises applying more bone screws by translating bone screws through further bone screw bores on the longitudinal plates. Optionally, the method may further comprise the steps of aligning slots in the connecting rods with the retaining pin bores and inserting retaining pins in the retaining pin bores and slots. Another optional embodiment of the invention involves translating one or more connecting rods having at least one end threaded into one or more threaded rod bores of one or more longitudinal plates and/or center plates. Yet a further embodiment comprises the step of aligning retaining pin holes in one or more connecting rods with retaining pin bores in one or more longitudinal plates and/or center plates and translating a retaining pin within the retaining pin bore and retaining pin hole. Preferably, the bone screws used prior to the step of applying the locking means are placed into and translated through bone screw bores which are not proximate to the connecting rods, i.e., bone screw bores in the longitudinal plates (or center plates) which are not adjacent to the recessed sections of the connecting rods and/or do not have the recessed section partially within the bone screw bores, then the bone screw bores adjacent to connecting rods and/or having connecting rods within may be used for the bone screws applied after the locking means is applied. In essence, the preferred method comprises initially anchoring longitudinal plates, and, optionally center plates as discussed below, into the bone, adjusting the longitudinal length of the bone plate assembly thereby moving the broken pieces of bone proximate to one another to set the bone in place and then compressing the longitudinal plates and optional center plate with the locking means.

The method may further comprise applying one or more center plates. In this embodiment, one center plate is placed over the second side of the break and additional connecting rods are used and inserted into the connecting rod bores on the second end of the center plate and then either additional center plates or the second longitudinal plate may be applied by translating the connecting rods from the second end of the center plate within the connecting rod bores of further center plates or the second longitudinal plate of the bone plate assembly. For example, the step of applying the center plate and additional connecting rods can be repeated over sides of broken bone by inserting the connecting rods, from the second end of the center plate into the first end of another center plate. The surgeon may either move and secure the longitudinal plate and center plate combination, or center plate and center plate combination either as each combination of pieces applied to the bone or after all pieces of the bone assembly are applied but before the locking means are applied. In any event, after the center plate is over the bone, the center plate may be secured to the bone by bone screws.

Methods wherein the connecting rods are permanently affixed to one or more longitudinal plates and/or center plates are also within the scope of the invention. Thus, rather than attaching longitudinal plates, then inserting connecting rods and then other longitudinal plates and/or center plate(s), the surgeon may attach a longitudinal plate and/or center plate with permanently affixed connecting rods then place a second center plate and/or a second longitudinal plate over the other side of the break and translate the permanently affixed connecting rods within the connecting rod bores of this adjoining center plate and/or longitudinal plate or may attach a first longitudinal plate and/or center plate to one side of the break and then translate the permanently affixed connecting rods of a second longitudinal plate and/or center plate within the connecting rod bores of the first longitudinal plate or center plate and then affix the second longitudinal plate or center plate on the bone on the opposite side of the break. In a preferred method, the surgeon affixes a connecting rod with either a threaded end or a retaining pin hole to either the forward rod bore or distal rod bore of a longitudinal plate or one end of a center plate leaving either the forward rod bore or distal rod bore of the longitudinal plate or end of a center plate vacant. The surgeon then affixes a connecting rod with either a threaded end or a retaining pin hole in the forward rod bore or distal rod bore of a second longitudinal plate or end of a center plate leaving the forward rod bore or distal rod bore of the second longitudinal plate and/or end of center plate vacant. The surgeon can then place the first longitudinal plate or center plate over one side of the broken bone and the second longitudinal plate or center plate over another side of the broken bone and translate the connecting rod from one longitudinal plate or center plate in the vacant rod bore of the opposite longitudinal plate or center plate. For example, when the bone plate assembly comprises a first longitudinal plate and a second longitudinal plate, a connecting rod would be affixed to the forward rod bore of the first longitudinal plate and a connecting rod would be affixed to the distal rod bore of the second longitudinal plate or the connecting rod would be affixed to the distal rod bore of the first longitudinal plate and the forward rod bore of the second longitudinal plate.

The surgeon moves the longitudinal plates and/or center plates to move the broken bones together for healing. The surgeon shall observe the bone pieces through the one or more observation windows, such as the observation windows defined by the connecting rods and all or some of 1) the second ends of the longitudinal plates, 2) the second end of a longitudinal plate and first end of a center plate, 3) the second end of a longitudinal plate and second end of a center plate and 4) the first end of a center plate and second end of a center plate. Further, the longitudinal plates and center plates described herein may be used with longitudinal plates and center plates which do not have the protrusion, such as those described in the inventors' co-pending applications for ADJUSTABLE BONE PLATE, which are the PCT application filed Oct. 24, 2005 and U.S. patent application Ser. No. 10/975,296 filed Oct. 28, 2004.

The assembled bone plates and the longitudinal plates and center plates of the invention may be constructed of any suitable biocompatible material, known to have sufficient structural strength and durability, such as stainless-steel, or titanium alloys. One example of such a material is ASTM F-136 titanium alloy (Ti 6AL-4V), which is a titanium alloyed with vanadium, or all ASTM materials. In addition the bone plate assembly and the longitudinal plates and center plates may be made of polymeric material such as PEEK (poly ethyl ethylketone), either separately or with other polymers or with other materials, such as reinforcing material. The material should have sufficient flex to mimic the micromotion of normal bone, to stimulate bone growth. Other materials which may be used are ceramic filled biocompatible polymers, or other biocompatible materials of sufficient strength to stabilize the bone during healing, or correct a fracture of the bone.

The bone plate assembly, the longitudinal plates and/or center plates may further comprise bioabsorbable drug delivery devices, such as implantable modular drug delivery devices. Examples of bioabsorbable drug delivery devices which may be used in the bone plate assembly, longitudinal plates and/or center plates are described in the co-pending application, U.S. Ser. No. 11/135,256 filed May 23, 2005, IMPLANTABLE PROSTHETIC DEVICES CONTAINING TIMED RELEASE THERAPEUTIC AGENTS, which is incorporated herein in its entirety by reference. Such devices, for example, may be placed within a dedicated bore, such as a drug delivery bore or pockets for drug or nano release, in one or more of the longitudinal plates and/or center plates, or within a bone screw bore or locking means bore of one or more of the longitudinal plates and/or center plates.

Bioabsorbable surgical fasteners or bone screws made from bioabsorbable materials may be used to apply the bone plate assembly, i.e. to apply the longitudinal plates and/or center plates, to the bone of a patient. For example, the materials described in the co-pending patent application, U.S. Ser. No. 11/025,231, filed Dec. 29, 2004, SURGICAL FASTENERS AND RELATED IMPLANT DEVICES HAVING BIOABSORBABLE COMPONENTS, which is incorporated herein in its entirety by reference, may be used for the bone screws and the bone screws may be the surgical fasteners described in this co-pending patent application.

What is claimed is:

1. A longitudinal plate for a bone plate assembly, the longitudinal plate comprising
a) an upper surface,
b) a lower surface,
c) a forward side,
d) a distal side,
e) a first end,
f) a second end having a recessed edge and a protrusion comprising an inward wall adjacent to the recessed edge, an end wall adjacent to the inward wall and an outward wall adjacent to the end wall,
g) one or more rod bores defined by rod bore walls and rod bore ends extending longitudinally from the second end to a point between the first end and the second end,
h) one or more bone screw bores defined by bone screw bore walls, and
i) optionally, a slit having a slit opening at the second end the slit being defined by a slit upper wall which extends longitudinally from the slit opening to a point between the second end and the first end of the longitudinal plate, a slit end and a slit lower wall which extends longitudinally from the slit opening to a point between the second end and the first end of the longitudinal plate.

2. The longitudinal plate of claim 1 wherein the first end comprises lateral curvature having an upper arc point about equidistant from the forward side and distal side.

3. The longitudinal plate of claim 1 further comprising a bump out.

4. The longitudinal plate of claim 1 further comprising a forward lobe and a distal lobe.

5. The longitudinal plate of claim 1 wherein the first end is comprised of a forward circular element and a distal circular element joined by a center section having curvature.

6. The longitudinal plate of claim 1 wherein one or more rod bores are threaded.

7. The longitudinal plate of claim 1 further comprising one or more retaining pin bores defined by retaining pin bore walls.

8. A center plate for a bone plate assembly, the center plate comprising
a) a top surface,
b) a bottom surface,
c) a forward longitudinal side,
d) a distal longitudinal side,
e) a first end,
f) a second end,
g) one or more recessed edges and one or more protrusions at the first end, the second end or a combination thereof, wherein the protrusion comprises an inward wall adjacent to the recessed edge, an end wall adjacent to the inward wall and an outward wall adjacent to the end wall,
h) two or more rod bores defined by at least rod bore walls,
i) one or more bone screw bores defined by bone screw bore walls,
j) optionally, a first slit having a first slit opening at the first end the first slit being defined by a first slit upper wall which extends longitudinally from the first slit opening to a point between the first end and the second end of the center plate, a first slit end and a first slit lower wall which extends longitudinally from the first slit opening to a point between the first end and the second end of the center plate and,
k) optionally, a second slit having a second slit opening at the second end the second slit being defined by a second slit upper wall which extends longitudinally from the second slit opening to a point between the second end and the first end of the center plate, a second slit end and a second slit lower wall which extends longitudinally from the second slit opening to a point between the second end and the first end of the center plate.

9. The center plate of claim 8 comprising two or more first end rod bores extending longitudinally from the first end to a point between the first end and the second end and two or more second end rod bores extending longitudinally from the second end to a point between the second end and the first end.

10. The center plate of claim 8 comprising a forward continuous rod bore and a distal continuous rod bore each extending longitudinally from the first end to the second end.

11. The center plate of claim 8 wherein one or more rod bores are threaded.

12. The center plate of claim 8 further comprising one or more retaining pin bores defined by retaining pin bore walls.

13. A bone plate assembly comprising:
a) one or more longitudinal plates having i) an upper surface, ii) a lower surface, iii) a forward side, iv) a distal side, v) a first end, vi) a second end, vii) one or more rod bores defined by rod bore walls and rod bore ends extending longitudinally from the second end to a point between the first end and the second end, viii) optionally, a slit having a slit opening at the second end the slit being defined by a slit upper wall which extends longitudinally from the slit opening to a point between the second end and the first end of the longitudinal plate, a slit end and a slit lower wall which extends longitudinally from the slit opening to a point between the second end and the first end of the longitudinal plate, and, ix) optionally, one or more locking means bores defined by locking means bore walls;
b) one or more connecting rods;
c) one or more locking means; and
d) one or more observation windows
wherein the second end of each longitudinal plate comprises a recessed edge and a protrusion comprising an inward wall adjacent to the recessed edge, an end wall adjacent to the inward wall and an outward wall adjacent to the end wall.

14. The bone plate assembly of claim 13 further comprising one or more retaining pins and one or more retaining pin bores defined by retaining pin bore walls within the longitudinal plates.

15. The bone plate assembly of claim 14 wherein the retaining pins comprise threaded sections.

16. The bone plate assembly of claim 13 further comprising one or more center plates having i) a top surface, ii) a bottom surface, iii) a forward longitudinal side, iv) a distal longitudinal side, v) a first end, vi) a second end, vii) two or more rod bores, defined by at least rod bore walls, viii) one or more bone screw bores defined by bone screw bore walls, ix) optionally, a first slit having a first slit opening at the first end the first slit being defined by a first slit upper wall which extends longitudinally from the first slit opening to a point between the first end and the second end of the center plate, a first slit end and a first slit lower wall which extends longitudinally from the first slit opening to a point between the first end and the second end of the center plate, x) optionally, a second slit having a second slit opening at the first end the second slit being defined by a second slit upper wall which extends longitudinally from the second slit opening to a point between the second end and the first end of the center plate, a second slit end and a second slit lower wall which extends longitudinally from the second slit opening to a point between the second end and the first end of the center plate and xi) optionally one or more locking means bores defined by locking means bore walls wherein the first end and second end of each center plate comprises a recessed edge and a protrusion comprising an inward wall adjacent to the recessed edge, an end wall adjacent to the inward wall and an outward wall adjacent to the end wall.

17. The bone plate assembly of claim 13 wherein the connecting rods have a first end and a second end with a recessed section at each end.

18. The bone plate assembly of claim 17 comprising a first longitudinal plate and a second longitudinal plate and forward connecting rod and a distal connecting rod wherein the first longitudinal plate comprises a forward rod bore and a distal rod bore and the second longitudinal plate comprises a forward rod bore and distal rod bore with the first end of the forward connecting rod within the forward rod bore of the first longitudinal plate and the second end of the forward connecting rod within the forward rod bore of the second longitudinal plate and the first end of the distal connecting rod within the distal rod bore of the first longitudinal plate and the second end of the distal connecting rod with the distal rod bore of the second longitudinal plate wherein the observation window is defined by the second end of the first longitudinal plate, the second end of the second longitudinal plate, the forward connecting rod and the distal connecting rod.

19. The bone plate assembly of claim 18 wherein the recessed section of the forward connecting rod at the first end and recessed section of the distal connecting rod at the first end are proximate to or within the at least one of bone screw bore of the first longitudinal plate and the recessed section of the forward connecting rod at the second end and recessed section of the distal connecting rod at the second end are proximate to or within the at least one bone screw bore of the second longitudinal plate.

20. The bone plate assembly of claim 18 further comprising i) a center plate having a first end and a second end, ii) a second forward connecting rod, and iii) a second distal connecting rod wherein the center plate has a one or more connecting rod bores and wherein p1 a) the second end of the forward connecting rod is within a rod bore at the first end of the center plate and the second end of the distal connecting rod is within a rod bore at the first end of the center plate;
b) the first end of the second forward connecting rod is within a rod bore at the second end of the center plate and the first end of the second distal connecting rod is within a rod bore at the second end of the center plate; and
c) the second end of the second forward connecting rod is within the forward rod bore of the second longitudinal plate and the second end of the distal connecting rod is within the distal rod bore of the second longitudinal plate
wherein a first observation window is defined by the second end of the first longitudinal plate, the first end of the center plate, the forward connecting rod and distal connecting rod and a second observation window is defined by the second end of the center plate, the second end of the second longitudinal plate, the second forward connecting rod and second distal connecting rod.

21. The bone plate assembly of claim 20 wherein the recessed section of the forward connecting rod at the first end and recessed section of the distal connecting rod at the first end are proximate to or within the at least one of bone screw bore of the first longitudinal plate, the center plate has, at least, a first end bone screw bore and a second end bone screw bore and the recessed section of the forward connecting rod at the second end and recessed section of the distal connecting rod at the second end are proximate to or within the first end bone screw bore of the center plate and the recessed section of the first end of second forward connecting rod and the recessed section of the first end of the second distal connecting rod are proximate to or within the second end bone screw bore of the center plate and the recessed section of the second end of the second forward connecting rod and the recessed section of the second end of the second distal connecting rod are proximate to or within one of the bone screw bores of the second longitudinal plate.

22. A method for setting a broken bone comprising the steps of
a) providing at least two longitudinal plates each longitudinal plate having an i) upper surface, ii) a lower surface, iii) a forward side, iv) a distal side, v) a first end, vi) a second end having a recessed edge and a protrusion comprising an inward wall adjacent to the recessed edge, an end wall adjacent to the inward wall and an outward wall adjacent to the end wall, vii) two or more rod bores defined by rod bore walls and rod bore ends, viii) optionally, a slit having a slit opening at the second end the slit being defined by a slit upper wall which extends longitudinally from the slit opening to a point between the second end and the first end of the longitudinal plate, a slit end and a slit lower wall which extends longitudinally from the slit opening to a point between the second end and the first end of the longitudinal plate, wherein at least one of the longitudinal plates comprises one or more locking means bores defined by locking means bore walls,
b) providing two or more connecting rods,
c) attaching a first longitudinal plate to a bone having a break on one side of the break by translating a bone screw through one of the bone screws bores of the first longitudinal plate and connecting the bone screw to the bone,
d) placing a second longitudinal plate over the bone on another side of a break in the bone of a patient,
e) translating connecting rods through rod bores in the first longitudinal plate, second longitudinal plate, or combinations thereof,
f) securing the second longitudinal plate to the bone by translating bone screws through one of the bone screw bores of the second longitudinal plate and connecting the bone screw to the bone,
g) moving the longitudinal plates while observing the location of the pieces of the broken bone through one or more observation windows,
h) providing locking means, and
i) translating the locking means through the locking means bores to apply the locking means.

23. The method of claim 22 wherein the inward wall of the protrusion on the first longitudinal plate is juxtaposed to the inward wall of the protrusion on the second longitudinal plate.

24. The method of claim 22 comprising the additional steps of
a) providing one or more center plates comprising i) a top surface, ii) a bottom surface, iii) a forward longitudinal side, iv) a distal longitudinal side, v) a first end, vi) a second end, vii) one or more rod bores defined by at least rod bore walls, viii) optionally a first slit having a first slit opening at the first end the first slit being defined by a first slit upper wall which extends longitudinally from the first slit opening to a point between the first end and the second end of the center plate, a first slit end and a first slit lower wall which extends longitudinally from the first slit opening to a point between the first end and the second end of the center plate, ix) optionally a second slit having a second slit opening at the second end the second slit being defined by a second slit upper wall which extends longitudinally from the second slit opening to a point between the second end and the first end of the center plate, a second slit end and a second slit lower wall which extends longitudinally from the second slit opening to a point between the second end and the first end of the center plate, x) optionally one or more locking means bores defined by locking means bore walls and xi) one or more bone screw bores defined by bone screw bore walls wherein the first end, second end or both the first end and second end comprise a recessed edge and a protrusion having an inward wall adjacent to the recessed edge, an end wall adjacent to the inward wall and an outward wall adjacent to the end wall, and
wherein i) the center plates are placed over and secured to pieces of broken bone between the longitudinal plates by translating the connecting rods within the connecting rod bores of the center plate, ii) the center plates are secured to the bone by translating bone screws through bone screw bores of the center plates and connecting the bone screw to the bone, iii) moving the longitudinal plates and/or center plates while observing the location of the pieces of the broken bone through one or more observation windows, iv) optionally providing locking means for the center plates and v) optionally translating the locking means through the locking means bores of the center plates to apply the locking means.

25. The method of claim 24 wherein the inward walls of adjacent longitudinal plates and center plates are juxtaposed.
